(19) United States
(12) Patent Application Publication
(10) Pub. No.: US 2009/0182383 A1
(43) Pub. Date: Jul. 16, 2009

(54) Bone fixation plate with anchor retaining member

(75) Inventors: Robert Prybyla, Two Woodhollow Trail, TX (US); Cory R. Schaffhausen, Salt Lake City, UT (US); John Bernero, Round Rock, TX (US)

Correspondence Address: MINTZ LEVIN COHN FERRIS GLOVSKY & POPEO, ONE FINANCIAL CENTER, BOSTON, MA, 02111, US

(73) Assignee: Amedica Corporation, Salt Lake City, UT (US)

(21) Appl. No.: 12/008,920

(22) Filed: Jan. 14, 2008

(51) Int. Cl. A61B 17/80; A61B 17/56

(52) U.S. Cl. 606/280; 606/71; 606/290

(57) ABSTRACT

Bone (e.g., spinal) fixation assemblies are provided that resist back out of associated bone anchors (e.g., screws). The bone fixation plate may house a bone anchor retaining member that permits partial passage of the bone anchor and subsequently resists back out of the bone anchor from the member and the bone fixation plate. In some embodiments, the bone fixation plate may include an attachable cover member that prevents the bone anchor retaining member from ejecting from the plate. In other embodiments, ejection of the anchor retaining member from the bone fixation plate may be prevented by walls integral to the plate.

### FIELD OF THE INVENTION

Embodiments of the present invention relate to bone fixation plates, and more particularly, to bone (e.g., spinal) fixation plates that resist back out of associated bone anchors (e.g., screws).

### BACKGROUND OF THE INVENTION

The spine is a flexible, multi-segmented column that supports upright posture in a human while providing mobility to the axial skeleton. The spine encases and protects vital neural elements while providing structural support for the body by transmitting the weight of the body through the pelvis to the lower extremities. The cervical spine exhibits a wide range of motion due to the orientation of its facets and the lack of supporting structures. The thoracic and lumbar regions of the spine also have a significant range of motion.

The spine is made up primarily of bone and intervertebral discs, which are surrounded by supporting ligaments, muscle, fascia, blood vessels, nerves, and skin. These elements are subject to a variety of pathological disturbances: inflammation, trauma, neoplasm, congenital anomalies, disease, etc. Trauma to the spine can play a large role in the etiology of neck and low back pain. For example, trauma frequently results in damage at the upper end of the lumbar spine, where the mobile lumbar segments join the less mobile dorsal spine. Excessive forces on the spine not only produce life-threatening traumatic injuries, but may contribute to an increased rate of degenerative change.

The cervical region of the spine comprises the seven most superior vertebrae of the spine, which begin at the base of the skull and end at the upper torso. Because the neck has a wide range of motion and is the main support for the head, the neck is extremely vulnerable to injury and degeneration.

Spinal fixation is a common method of treating spinal disorders, fractures, and degeneration. One common device used for spinal fixation is the bone fixation plate, which is typically used in conjunction with a graft device placed between the vertebral bodies. Generally, there are two types of spinal plates: (i) constrained plates and (ii) semiconstrained plates. Generally, a constrained plate completely immobilizes the vertebrae and does not allow for graft settling. In this instance, the plate itself carries a significant portion of the loading. Constrained plates are useful, for example, in patients with highly unstable anatomy, such as with a vertebrectomy, or in patients with little chance of bone growth, such as cancer patients. In contrast, a semiconstrained plate is dynamic and allows for a limited degree of graft settling through micro-adjustments made between the plate and bone screws attaching the plate to the spine. The operation of the semiconstrained plate stimulates bone growth because the loading is transferred through the graft. Each type of plate has its own advantages depending upon the anatomy and age of the patient, and the results desired by the surgeon.

A typical bone fixation plate includes a relatively flat, rectangular plate having a plurality of apertures formed therein. A corresponding plurality of bone screws may be provided to secure the bone fixation plate to the vertebrae of the spine. A common problem associated with such a bone fixation plate is the tendency for bone screws to become dislodged from the bone and “back out” from the plate, thereby causing the plate to loosen and the screws to protrude from the plate. For example, in a typical anterior cervical fusion surgery, the carotid sheath and sternocleidomastoid muscles are moved laterally and the trachea and esophagus are moved laterally in order to expose the cervical spine. The cervical plate is designed to lie on the anterior face of the spine, dorsal to the esophagus. Due to its relative location to the esophagus and other connective tissue, if the bone screw securing the plate to the cervical spine backs out, the bone screw could pierce the esophagus, causing not only pain and infection, but also posing a serious risk of death to the patient. Bone fixation plates with large anterior-posterior profiles (e.g., thickness) can also make it difficult for the patient to swallow post-surgery.

In view of the foregoing, it would be desirable to provide bone fixation assemblies that resist back out of associated bone anchors.

### SUMMARY OF THE INVENTION

Embodiments of the present invention relate to bone plating systems that resist back out of associated bone anchors.

In an aspect, a bone fixation apparatus is provided that includes a bone fixation plate, a bone anchor (e.g., bone screw), and a bone anchor retaining member. The bone fixation plate includes a top surface, a bottom surface, and at least one aperture between the top surface and the bottom surface for permitting partial passage of a bone anchor through the plate. The bone fixation plate additionally includes a cavity formed between the top surface and the bottom surface, where the cavity at least partially intersects (e.g., is coaxial with) the aperture. The bone anchor retaining member is housed at least partially within the cavity, and is configured to transition from a first position to a second position in response to interaction with the bone anchor. In the first (e.g., open) position, the bone anchor retaining member permits partial passage of the bone anchor through the member. In the second (e.g., closed) position, the member resists back out of the bone anchor.

In some embodiments, the bone anchor retaining member may be formed generally in the shape of a ring. In one embodiment, in the first position the bone anchor retaining member may have a generally convex shape (e.g., conical). In the second position, the bone anchor retaining member may have a generally concave shape (e.g., inverse conical). In another embodiment, the bone anchor retaining member may have a generally convex shape in both the first and second positions.

In some embodiments, the bone anchor retaining member may include multiple surfaces (e.g., tabs) extending towards a center of the aperture formed within the bone fixation plate. In the second position, a distance between the plurality of surfaces may be less than a distance between the plurality of surfaces in the first position.

In still other embodiments, the member may transition from the first position to the second position via a third position, wherein in the third position a distance between the plurality of surfaces is less than both the distance between the plurality of surfaces in the first position and the distance between the plurality of surfaces in the second position.

In some embodiments, the bone anchor retaining member may be flat (e.g., a flat ring-shaped member) both before and after partial passage of the bone anchor through the member. In response to interaction of the retaining member with the bone anchor, the retaining member may deform in the direction of advancement of the anchor, thus increasing the size of an internal diameter of the member such that it allows partial passage of the anchor. The retaining member may then return to its original, flat configuration in order to resist back out of the anchor.

In some embodiments, the bone fixation plate includes a generally part-spherical surface adjacent to the at least one aperture and the top surface, for example, for multi-angular articulation with a complimentary part-spherical surface of the bone anchor.

In some embodiments, the bone fixation plate comprises a generally part-cylindrical surface adjacent to the at least one aperture and the bottom surface.

In some embodiments, when the bone anchor is advanced fully into the plate, the width of the aperture in the bone fixation plate may be substantially equal to a width of an adjacent portion of the bone anchor. This may constrain movement of the fixation plate subsequent to the procedure (e.g., rigid fixation). In other embodiments, the width of the aperture may be greater than the width of the adjacent portion of the bone anchor, which may allow for dynamic movement of the fixation plate at an implantation site.

In some embodiments, the width of the cavity in the bone fixation plate may be substantially equal to a width of the bone anchor retaining member, which may constrain movement of the fixation plate. In other embodiments, the width of the cavity may be greater than the width of the bone anchor retaining member, which may allow for dynamic movement of the fixation plate at an implantation site.

In still other embodiments, the bone fixation plate may include multiple aperture and/or cavity sizes, which may allow the same plate to be used for both rigid and dynamic fixation, at the option of the surgeon. For example, the same bone fixation plate may include a set of apertures and/or cavities configured for rigid fixation, and an independent set of apertures and/or cavities configured for dynamic fixation.

In some embodiments, the bone anchor may be a bone screw that includes a head, a shoulder in communication with the head, a groove in communication with the shoulder, and a threaded shank in communication with the groove. The head may be configured for articulation with the bone fixation plate. The shoulder may be configured to contact the bone anchor retaining member when the member is in the first position. The groove may be configured to receive the bone anchor retaining member when the member is in the second position. A width of the shoulder may be greater than a width of the threaded shank.

In some embodiments, the bone anchor retaining member may be formed from an elastic material.

In still other embodiments, the bone anchor retaining member may form a plurality of peaks and valleys in top and bottom surfaces of the member. The retaining member may be configured to transition from a first, convex position to a second, convex position in response to interaction of the member with the bone anchor. In the first position, the bone anchor retaining member may permit partial passage of the bone anchor through the member. In the second position, the retaining member may resist back out of the bone anchor.

In some embodiments, the bone fixation plate may include an attachable cover member (e.g., ring-shaped cover member) that is substantially co-axial with the cavity and the ring-shaped member and that is configured to prevent the ring-shaped member from ejecting from the cavity. In some embodiments, the cover member forms the bottom surface of the bone fixation plate.

In another aspect, a method for bone fixation is provided. The method includes advancing a bone anchor (e.g., screwing a bone screw) through a bone fixation assembly and into bone, and encountering resistance to the advancing before the bone anchor is advanced fully into the bone, where the resistance is attributable to the bone fixation assembly. The method additionally includes advancing the bone anchor into the bone to overcome the resistance, and subsequent to the further advancing, resisting back out of the bone anchor from the bone fixation assembly. In some embodiments, encountering resistance to the advancing includes contacting a portion of the bone fixation assembly with a shoulder of the bone anchor.

In still another aspect, a bone fixation apparatus is provided that includes means housed within a bone fixation plate for resisting advancement of a bone anchor before the bone anchor is advanced fully into the bone. Upon further advancement of the bone anchor through the bone fixation plate, the means housed within the bone fixation plate further comprises means for resisting back out of the bone anchor from the bone fixation plate.

### BRIEF DESCRIPTION OF THE DRAWINGS

For a better understanding of the present invention, including the various objects and advantages thereof, reference is made to the following detailed description, taken in conjunction with the accompanying illustrative drawings, in which like reference characters refer to like parts throughout, and in which:

FIG. 1A is a perspective view of a bone fixation assembly including a bone fixation plate, at least one bone anchor (e.g., screw), and at least one member for retaining the bone anchor within the plate, according to some embodiments of the present invention;

FIG. 1B is a perspective view of another embodiment of a bone anchor in accordance with the present invention;

FIG. 1C is a top view of the bone anchor of FIG. 1B;

FIG. 2 is an enlarged, cross-sectional view of the bone fixation assembly of FIG. 1A, in which the bone anchor retaining member is in an open position for permitting passage of the anchor through the fixation plate and into bone;

FIG. 3 is an enlarged, cross-sectional view of the bone fixation assembly of FIG. 1A, in which the bone anchor retaining member is in a closed position for preventing back out of the anchor from the plate;

FIG. 4 is an enlarged, perspective view of the anchor retaining member of FIG. 1A in the open position;

FIG. 5 is an enlarged, perspective view of the anchor retaining member of FIG. 1A in the closed position;

FIG. 6 is a diagram that illustrates changes to the width of an opening in the anchor retaining member of FIG. 1A due to a transition of the member from the open position to the closed position, according to some embodiments of the present invention;

FIG. 7 shows placement of an anchor retaining member within a bone fixation plate according to an embodiment of the present invention;

FIG. 8 is an enlarged, perspective view of another embodiment of an anchor retaining member in the open position according to the present invention;

FIG. 9 is an enlarged, perspective view of the anchor retaining member of FIG. 8 in the closed position;

FIG. 10A is a perspective view of another embodiment of a bone fixation assembly in accordance with the present invention;

FIG. 10B is an exploded view of the bone fixation assembly of FIG. 10A;

FIG. 11 is an enlarged, perspective view of the anchor retaining member of FIGS. 10A and 10B; and

FIGS. 12A-C are cross-sectional views showing deformation and reformation of the anchor retaining member of FIG. 11 in response to interaction with a bone anchor.

### DETAILED DESCRIPTION OF THE INVENTION

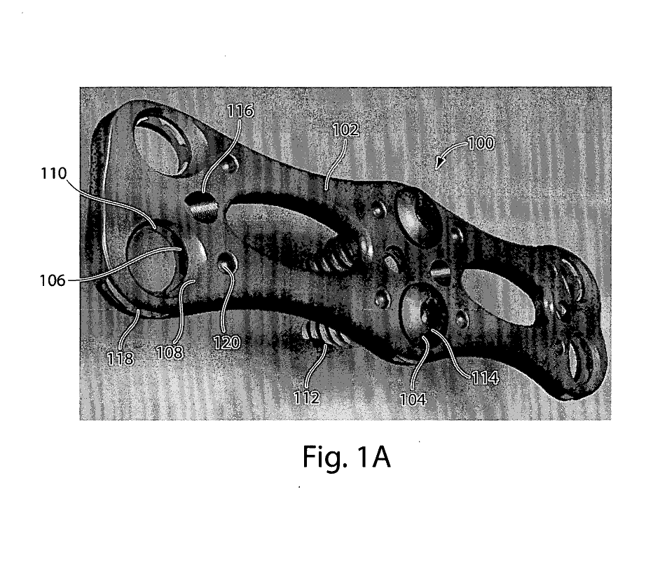

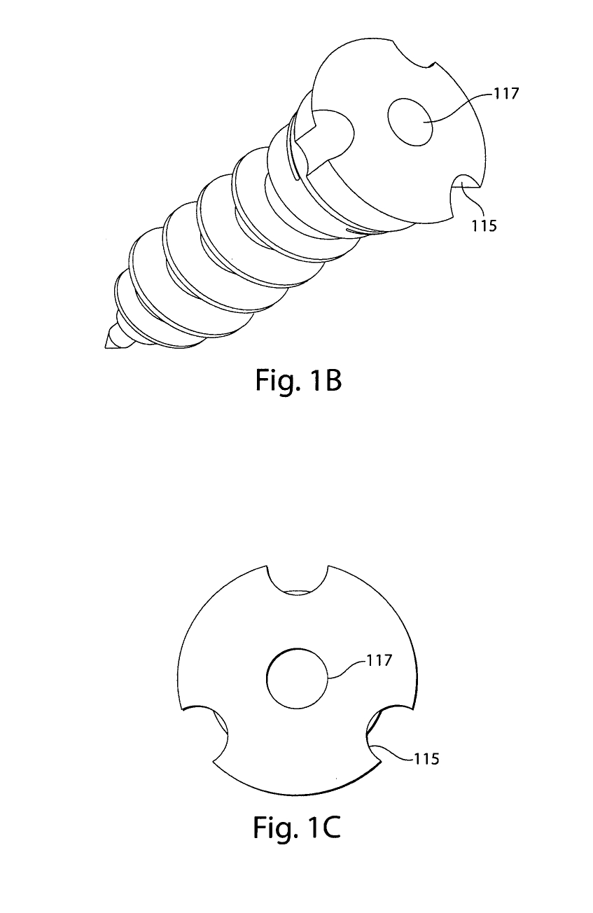

FIG. 1A is a perspective view of a bone fixation assembly 100 according to some embodiments of the present invention. Assembly 100 includes bone fixation plate 102, bone anchors (e.g., screws) 104, and one or more (e.g., six) installations of anchor retaining member 106. For example, FIG. 1A shows a two-level bone fixation plate 102 that is configured to span across and fixate three vertebrae of the cervical spine. In this embodiment, plate 102 includes six anchor retaining members 106. In other embodiments, single-level plates and other multi-level plates may be provided with different numbers of anchor retaining members 106.

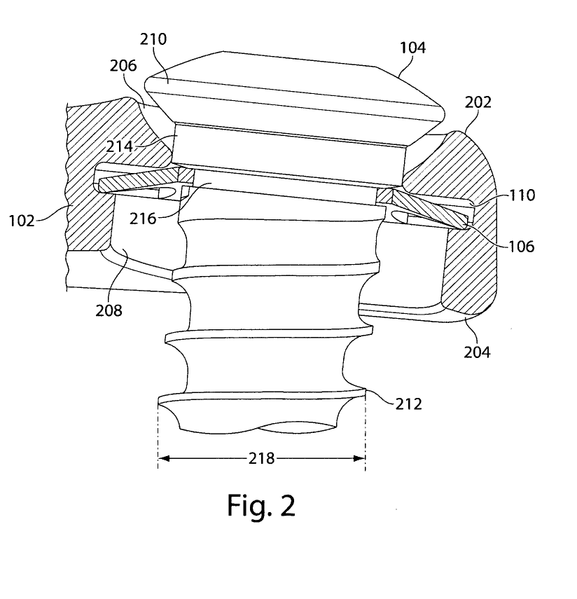

Bone fixation plate 102 may form a plurality of apertures 108 (e.g., six circular or part-circular apertures in the embodiment of FIG. 1A) for permitting passage of a corresponding plurality of bone anchors 104 through plate 102 and into bone. Bone fixation plate 102 may additionally form one or more cavities 110 (e.g., cylindrical or part-cylindrical cavities) for housing a corresponding one or more anchor retaining members 106 (e.g., rings). For example, in FIG. 1A, bone fixation plate 102 forms a cavity 110 adjacent to each aperture 108. In other embodiments, only a portion of apertures 108 may have cavities 110 and anchor retaining members 106 adjacent thereto. As described more fully in connection with FIGS. 2-6, the relative positions of aperture 108 and cavity 110 and the configuration of anchor retaining member 106 and bone anchor 104 may cause member 106 to move from an open position to a closed position responsive to passage of bone anchor 104 through aperture 108.

Bone anchors 104 may be configured at their distal ends 112 for self-tapping or self-drilling. Proximal ends 114 (heads) of bone anchors 104 may include a recess (e.g., having a non-circular cross-sectional shape) and/or other features for receiving a complimentary tip of a surgical tool. For example, in the embodiment of FIG. 1A, bone anchor 104 includes a hex-shaped feature formed within its proximal end 114. FIGS. 1B and 1C show another embodiment of a bone anchor that includes multiple (e.g., three) prongs 115 along the perimeter of its proximal end and a central, non-circular recess 117 for receiving a surgical tool. In some embodiments, the bone anchor shown in FIGS. 1B and 1C may be similar to or the same as the bone anchor of FIG. 1A in all other respects.

Bone fixation plate 102 may also form apertures 116 and slots 118 in communication with cavities 110, and indentations 120. Apertures 116 and indentations 120 may be configured for attachment to a delivery tool that positions plate 102 at an appropriate implantation site. In some embodiments, apertures 116 and/or slots 118 may permit access to anchor retaining members 106 to permit passage of a tool that re-opens/re-inverts members 106 from the closed position to the open position. In some embodiments, to re-open (unlock) member 106 after bone anchor 104 is screwed into place, bone anchor 104 may be unscrewed or otherwise backed out (e.g., about 1 thread turn), followed by introduction of a tool through a slot 118 to invert/open member 106. In other embodiments, member 106 may be re-opened by a tool that is secured to plate 102 and bone anchor 104. The tool may incorporate a member (e.g., trigger actuated member) that pushes against the top side of plate 102 while the tool is secured to and exerting an upward force on anchor 104, thus forcing anchor 104 upwardly and causing retaining member 106 to invert and open.

Bone fixation assembly 100 and its various components may be made from any suitable material or combination of materials. For example, in some embodiments, all of components 102, 104, and 106 may be made from titanium, stainless steel, and/or other biocompatible metal(s). In other embodiments, one or more (e.g., all) of components 102, 104, and 106 may be made from a polymer or one or more biocompatible ceramic(s), such as the doped silicon nitride ceramic described in commonly-owned U.S. Pat. No. 6,881,229, which is hereby incorporated by reference herein in its entirety. The one or more materials used for anchor retaining member 106 preferably have an elastic property.

In some embodiments, bone fixation plate 102 may have a lordotic curvature that corresponds to a lordotic curvature of the human cervical spine. For example, an anterior face of plate 102 may be contoured and rounded so as to reduce or eliminate irritation of the esophagus and the surrounding tissues.

In some embodiments, bone fixation plate 102 may be configured to promote bone ingrowth to the plate. For example, in some embodiments, at least a portion of bone fixation plate 102 may be made from a porous material, such as the porous silicon nitride ceramic described in commonly-owned U.S. Pub. Appln. No. 20050049706, which is hereby incorporated by reference herein in its entirety. Alternatively or additionally, one or more bone contacting surfaces of bone fixation plate 102 may be roughened, for example, by mechanical blasting and/or plasma spraying with metal particles of one or more sizes.

In some embodiments, bone fixation plate 102 may be coated with a bio-active material having an osteoconductive property, such as hydroxyapatite or a calcium phosphate material. Alternatively or additionally, bone fixation plate 102 may carry one or more therapeutic agents, for example, for enhancing bone fusion and ingrowth. Examples of such therapeutic agents include natural or synthetic therapeutic agents such as bone morphogenic proteins (BMPs), growth factors, bone marrow aspirate, stem cells, progenitor cells, antibiotics, and other osteoconductive, osteoinductive, osteogenic, bio-active, or any other fusion enhancing material or beneficial therapeutic agent. In some embodiments, bone anchor 104 and/or anchor retaining member 106 may be porous, roughened, and/or coated with one or more bio-active and/or therapeutic materials.

FIG. 2 is an enlarged, cross-sectional view of bone fixation plate 102, bone anchor 104, and anchor retaining member 106, in which member 106 is in an open position within cavity 110. Cavity 110 is located between (e.g., about midway between) top surface 202 and bottom surface 204 of bone fixation plate 102. Cavity 110 may at least partially intersect aperture 108 formed by surfaces 206 and 208 of plate 102. For example, cavity 110 may be coaxial with aperture 108, although cavity 110 may be wider than aperture 108 (e.g., the portion of aperture 108 formed by the innermost portion of surface 206). Surface 206 of bone fixation plate 102 may form a part-spherical seat configured for multi-angular articulation with a complimentary part-spherical surface 210 of bone anchor 104. Surface 208 may be generally cylindrical and may have a diameter that is wider than the threads of shank 212 of bone anchor 104. Bone anchor 104 may include shoulder 214, and groove 216 for receiving anchor retaining member 106 when member 106 is in a closed position.

In the open position, anchor retaining member 106 may form an opening having a width greater than width 218 of threaded shank 212, where width 218 is equal to the major diameter of the threads. In other embodiments, the width of the opening within anchor retaining member 106 in the open position may be greater than (e.g., only slightly greater than) the width of the minor diameter of the threads, such that the screw is threaded through the anchor retaining member 106. The width of aperture 108 formed by surfaces 206 and 208 may also be greater than width 218. This may allow threaded shank 212 to pass through bone fixation plate 102 and anchor retaining member 106 and into bone. Shoulder 214 of bone anchor 104 may be wider than the opening in member 106, which may cause shoulder 214 to contact member 106 as anchor 104 is screwed into the bone. For example, shoulder 214 may contact anchor retaining member 106 just prior (e.g., less than about 1 screw turn prior) to bone anchor 104 being fully screwed into the bone. Additional screwing of bone anchor 104 into the bone after the occurrence of such contact may cause anchor retaining member 106 to transition from the open position to the closed position. In some embodiments, providing anchor retaining member 106 within plate 102 causes member 106 to interact with shoulder 214 and not the top of screw head 114 (FIG. 1A). Advantageously, this may prevent member 106 from, for example, interfering with a tool that screws and/or unscrews bone anchor 104 into and/or out of plate 102.

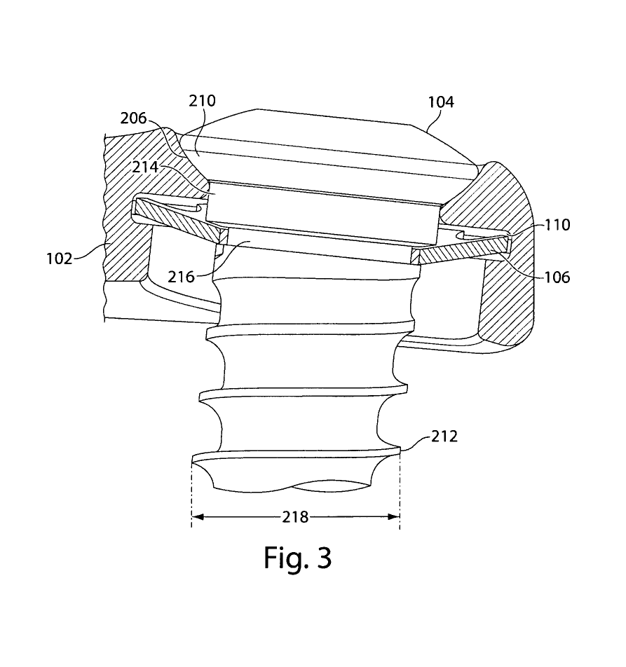

FIG. 3 is an enlarged, cross-sectional view of the bone fixation assembly of FIG. 1A, in which bone anchor retaining member 106 is in the closed (locked) position. In the closed position, the width of the opening in anchor retaining member 106 may be reduced to less than a width of threaded shank 212 (e.g., less than major diameter 218 or the minor diameter of the threads), which may prevent threaded shank 212 from backing out of member 106 and thus member 102. For example, the width of the opening may be slightly greater than the width of groove 216 formed in bone anchor 104. In some embodiments, the width of the inner most part of surface 206 in bone fixation plate 102 may be approximately equal to the width of shoulder 214 of bone anchor 104. This may prevent lateral movement of bone anchor 104 within plate 102 and cause rigid fixation between surface 206 of plate 102 and surface 210 of anchor 104. In other embodiments, the width of the inner most part of surface 206 may be greater than the width of shoulder 214. This may allow for movement of bone anchor 104 within plate 102 and dynamic articulation of surfaces 206 and 210.

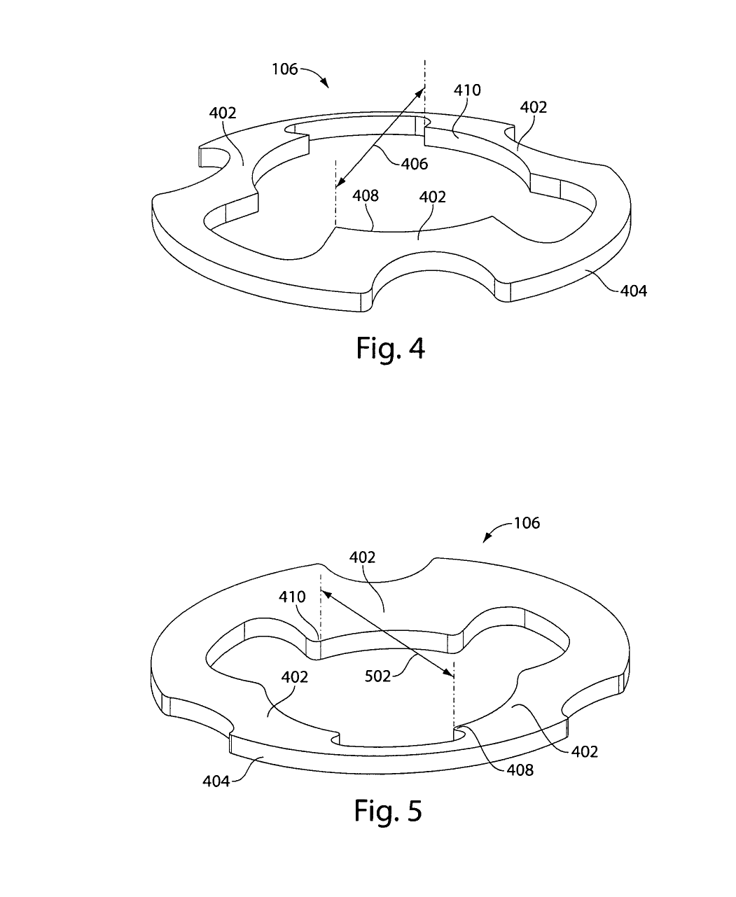

FIG. 4 is an enlarged, perspective view of anchor retaining member 106 in the open position. In some embodiments, member 106 may be generally ring-shaped. Member 106 may include one or more (e.g., three) tabs 402 that form the opening (e.g., part-circular opening) allowing for passage of threaded shank 212 (FIG. 2). Tabs 402 may slope upwardly (e.g., conically) from outer surface 404 of member 106 towards the center of member 106. Thus, in the open position, member 106 may be convex. In the embodiment of FIG. 4, anchor retaining member 106 is a single piece of solid, although elastic, construction. In other embodiments, member 106 may be formed from multiple pieces. In the open position, the opening formed by tabs 402 may have width 406 (shown generally as the distance between surfaces 408 and 410 of tabs 402), which may be greater than major diameter 218 of threaded shank 212 or greater than the minor diameter of the threads. Tabs 402 may be configured to contact shoulder 214 of bone anchor 104 and to resist further advancement of anchor 104 into the bone, prior to surface 210 of anchor 104 being seated within surface 206 of bone fixation plate 102. Such resistance may be overcome by further advancement (e.g., screwing) of bone anchor 104 into the bone.

FIG. 5 is an enlarged, perspective view of anchor retaining member 106 in the closed position. Tabs 402 may slope downwardly (e.g., inverse conically) from outer surface 404 of member 106 towards the center of member 106. Thus, in the closed position, member 106 may be concave. The opening formed by tabs 402 may have reduced width 502, which may be less than both width 406 formed by tabs 402 in the open position and a width of threaded shank 212 (e.g., less than major diameter 218 or the minor diameter of the threads). Width 502 is shown as the reduced distance between surfaces 408 and 410 of tabs 402. This reduced inner dimension of member 106 may prevent threaded shank 212 from backing out of member 106 and thus plate 102.

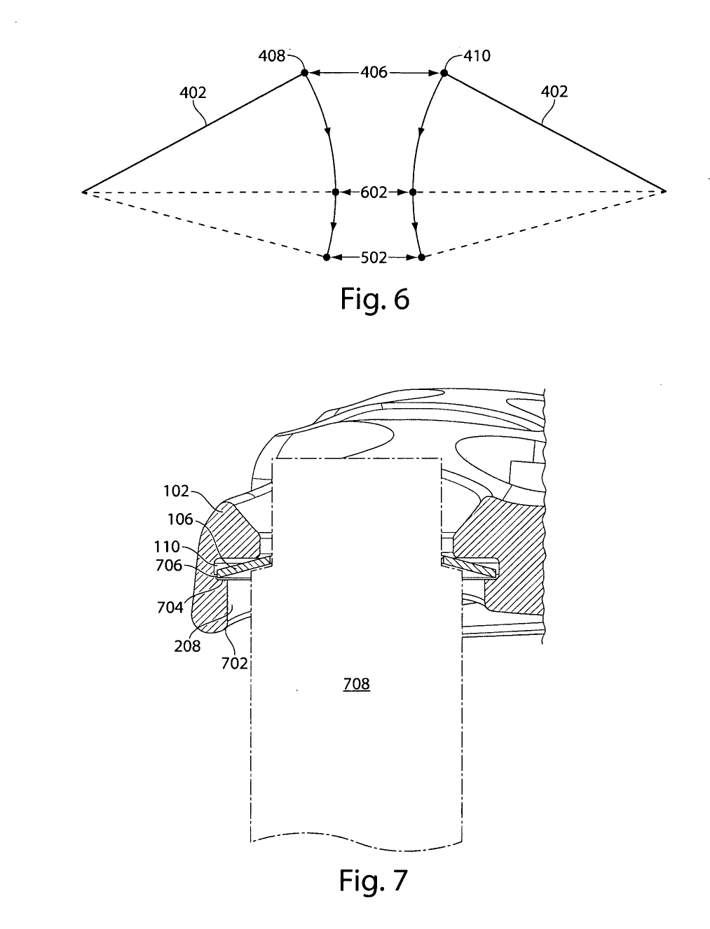

FIG. 6 is a diagram that illustrates changes to the width of the opening in anchor retaining member 106 during a transition of the member from the open position to the closed position. More specifically, FIG. 6 is an enlarged, side view of the path of surfaces 408 and 410 of tabs 402 during the transition, where the distance between surfaces 408 and 410 represents the width of the opening. In some embodiments, the paths traversed by surfaces 408 and 410 may form an hourglass-shape. In the open position, tabs 402 may form a generally convex shape and surfaces 408 and 410 may be separated by distance 406. Distance 406 may be greater than a width of threaded shank 212 (e.g., greater than major diameter 218 or the minor diameter of the threads). At an intermediate position along the paths, tabs 402 may be generally parallel and surfaces 408 and 410 may be separated by distance 602. Distance 602 may be less than the width of threaded shank 212, and substantially equal to or greater than the width of groove 216 in bone anchor 104. In the closed position, tabs 402 may form a generally concave shape and surfaces 408 and 410 may be separated by distance 502. Distance 502 may be less than a width of threaded shank 212 (e.g., less than major diameter 218 or the minor diameter of the threads) but greater than distance 602. An elastic property of retaining member 106 may be sufficiently low enough to allow member 106 to invert (e.g., from the open to the closed position) responsive to the mechanical force exerted by the interaction of the anchor and the plate, while being sufficiently high enough to resist inverting responsive to the physiological forces imposed upon the assembly in vivo.

FIG. 7 shows placement of an anchor retaining member 106 within cavity 110 of bone fixation plate 102 during manufacturing according to an embodiment of the present invention. As shown, retaining member 106 may be assembled into plate 102 through the bottom side of the plate. Bore 208 may include a lead-in angle or radius 702 to guide member 106 into cavity 110. Bore 208 may be slightly smaller than the outside diameter of retaining member 106, and may have a defined edge 704 to ensure that the retaining member 106 cannot easily slip out. Retaining member 106 may utilize adjacent surface 706 to provide opposing force when shoulder 214 (FIG. 2) engages member 106. Tool 708 may use a center bore of retaining member 106 to guide member 106 into cavity 110. To allow passage of member 106 through bore 208, the surfaces of plate 102 that form bore 208 may apply axial force to member 106, thus causing member 106 to collapse slightly. When retaining member 106 reaches cavity 110, an elastic property of member 106 may cause it to expand into and become captured within cavity 110 of plate 102.

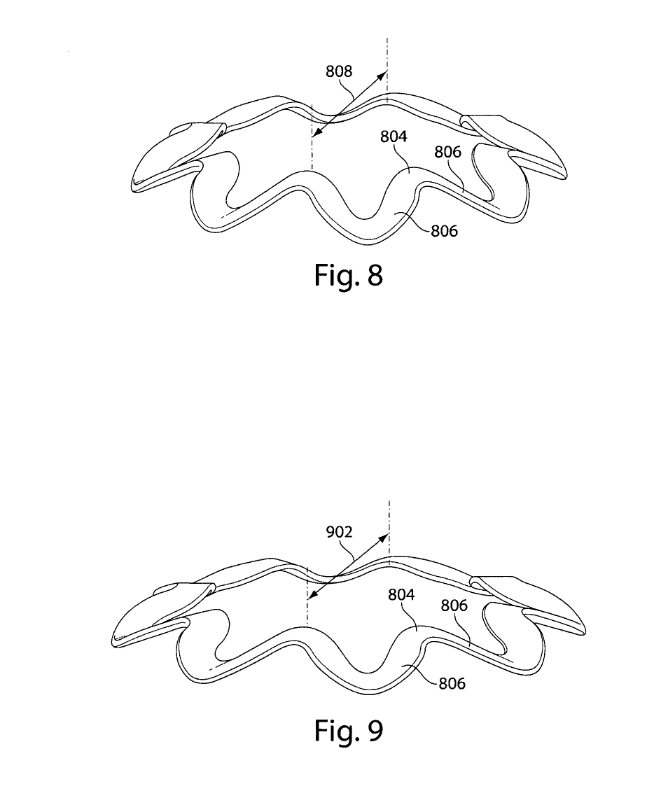

FIG. 8 is an enlarged, perspective view of another embodiment of an anchor retaining member 802 in an open position according to the present invention. Anchor retaining member 802 may include a plurality of tabs 802 (e.g., 8 tabs) that form the opening allowing for passage of threaded shank 212 (FIG. 2). Each of tabs 804 may be formed by a pair of arms 806 that slope upwardly from an outer surface toward the center of the opening. This upwardly sloping characteristic of arms 806 may form a generally convex, crinkle-shaped surface with a plurality of peaks and valleys in the top and bottom surfaces of anchor retaining member 802. In the embodiment of FIG. 8, anchor retaining member 802 is a single piece of solid, although elastic, construction. In other embodiments, member 802 may be formed from multiple pieces. In the open position, the opening formed by opposed tabs 804 may have width 808, which may be greater than width 218 of threaded shank 212. Tabs 804 may be configured to contact shoulder 214 of bone anchor 104 and to resist further advancement of anchor 104 into the bone, prior to surface 210 of anchor 104 being seated within surface 206 of bone fixation plate 102. Such resistance may be overcome by further advancement (e.g., screwing) of bone anchor 104 into the bone.

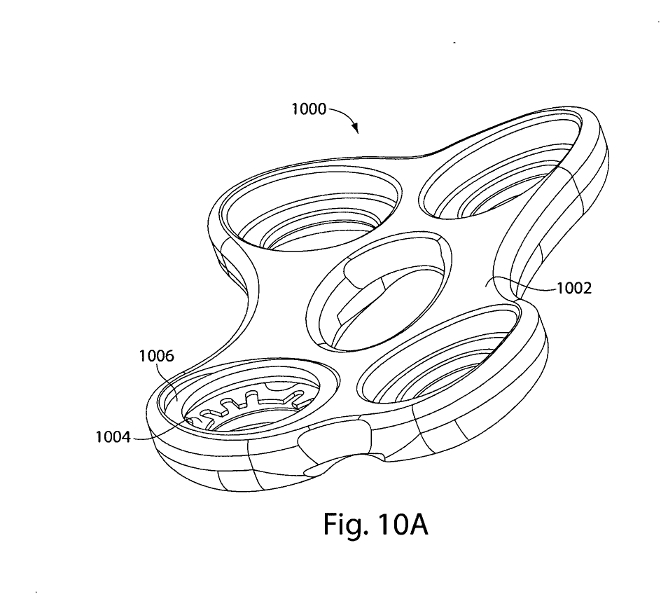

FIG. 9 is an enlarged, perspective view of the anchor retaining member of FIG. 8 in the closed position. Generally, in the closed position, anchor retaining member 802 may have a more flattened, albeit still generally convex, topology. Arms 806 that support tabs 804 may still slope upwardly from the outer surface of member 802 towards the center of member 106, although the magnitude of the slope may be reduced. The opening formed by tabs 804 may have reduced width 902, which may be less than both width 808 formed by tabs 804 in the open position and width 218 of threaded shank 212. Width 902 is shown as the reduced distance between two opposed tabs 804. This reduced inner dimension of member 802 may prevent threaded shank 212 from backing out of member 802 and thus plate 102. In some embodiments, the procedure for inserting member 802 into bone plate 102 during manufacturing may be the same as the procedure described above in connection with FIG. 7.

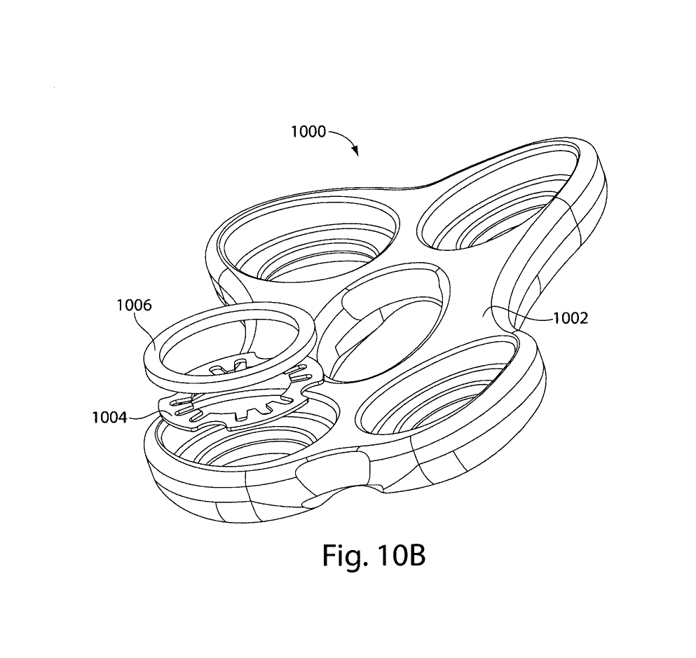

FIG. 10A is a perspective view of another embodiment of a bone fixation assembly 1000 in accordance with the present invention. FIG. 10B is an exploded view of the bone fixation assembly of FIG. 10A. Bone fixation assembly 1000 includes bone fixation plate 1002, one or more (e.g., four) anchor retaining members 1004, one or more (e.g., four) cover members 1006 that may be substantially co-axial with members 1004, and one or more bone anchors (not shown). In this example, assembly 1000 is a single-level assembly that spans two vertebral bodies, although other multi-level configurations may be provided in other embodiments. Each cover member 1006 may prevent a corresponding retaining member 1004 from ejecting from a cavity within bone fixation plate 1002. Generally, once an anchor retaining member 1004 is loaded into the cavity formed within plate 1002 and cover member 1006 is fixed to the assembly, the cross-section of assembly 1000 may be similar to the cross-sections shown in FIGS. 2 and 3, with the exception that member 1004 may be flat when viewed from the side. Each cover member 1006 may be fixed within bone fixation plate 1002 by any suitable approach including, for example, welding, bonding, and/or a threaded connection. In other embodiments, retaining member(s) with different cross-sectional characteristics (e.g., the retaining members shown in FIGS. 4, 5, 8, and 9) may be used in connection with bone fixation plate 1002 and cover member(s) 1006. In the embodiment shown in FIGS. 10A and 10B, cover member 1006 forms the bottom surface of bone fixation plate 1002 once it is fixed to the plate. In other embodiments, cover member 1006 may form the top surface of the bone fixation plate.

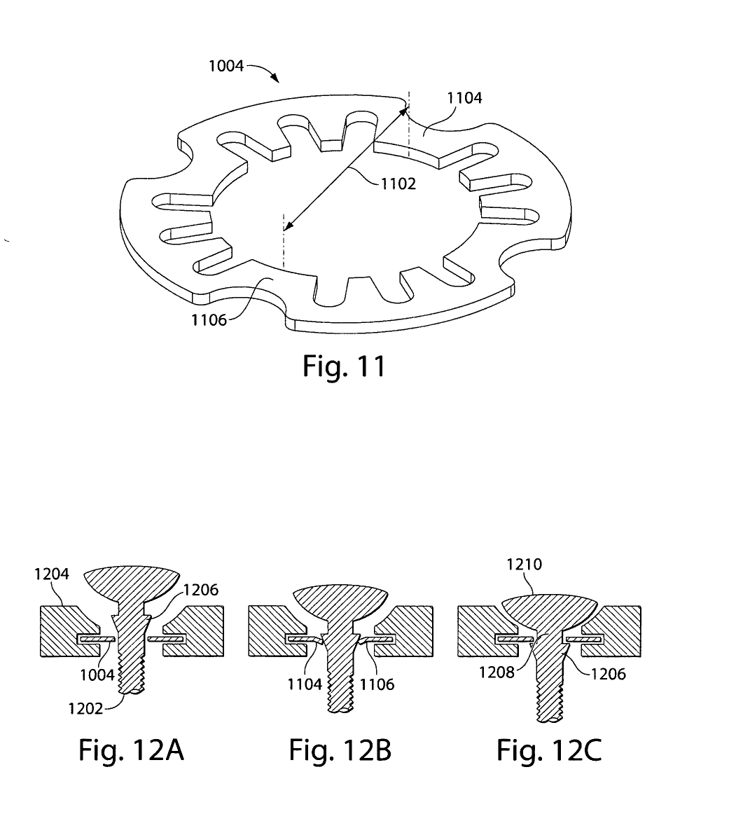

FIG. 11 is an enlarged, perspective view of anchor retaining member 1004. Anchor retaining member 1004 may be flat (e.g., a flat ring-shaped member) when viewed from the side both before and after partial passage of the bone anchor through the member. In response to interaction of retaining member 1004 with the bone anchor, retaining member 1004 may deform in the direction of advancement of the anchor, thus increasing the size of an internal diameter 1102 of the member (shown generally as the distance between tabs 1104 and 1106) until it allows partial passage of the anchor. Retaining member 1004 may then return to its original, flat configuration in which it resists back out of the anchor. In the embodiment of FIG. 11, anchor retaining member 1004 is a single piece of solid, although elastic, construction. In other embodiments, member 1004 may be formed from multiple components.

FIGS. 12A-C are cross-sectional views showing deformation and reformation of anchor retaining member 1004 in response to interaction with bone anchor 1202. In the embodiment shown in FIGS. 12A-C, anchor retaining member 1004 is held in place by walls that are integral to plate 1204 (e.g., a plate similar to if not the same as plate 102 (FIG. 1A)), although placement of member 1004 within other types of bone fixation plates is of course possible (e.g., the plate shown FIG. 10A). As shown in FIG. 12A, anchor retaining member 1004 may be flat prior to interaction with shoulder 1206 of bone anchor 1202. In some embodiments, shoulder 1206 may have a ramped (e.g., conical) configuration with its diameter increasing in the direction opposite to the direction of advancement of the anchor. FIG. 12B shows that interaction of shoulder 1206 with the tabs (e.g., including but not necessarily limited to tabs 1104 and 1106) of member 1004 causes the tabs to deform in the direction of advancement of the anchor. FIG. 12C shows that anchor retaining member 1004 may return to its flat configuration once shoulder 1206 passes through tabs 1104 and 1106. The tabs may then rest within groove 1208 of bone anchor 1202, which may be formed in bone anchor 1202 between shoulder 1206 and anchor head 1210. The tabs of member 1004 may resist back out of bone anchor 1202 when they are positioned within groove 1208.

Thus it is seen that bone fixation plates with anchor retaining members are provided. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed is:

1. A bone fixation apparatus, comprising:
a bone fixation plate comprising a top surface, a bottom surface, and at least one aperture between the top surface and the bottom surface for permitting partial passage of a bone anchor through the plate, the bone fixation plate further comprising a cavity formed between the top surface and the bottom surface and at least partially intersecting the aperture; and
a bone anchor retaining member within the cavity, the bone anchor retaining member configured to transition from a first position to a second position in response to interaction of the member with the bone anchor, wherein in the first position the bone anchor retaining member permits partial passage of the at least one bone anchor through the member and in the second position the member resists back out of the bone anchor.

2. The apparatus of claim 1, wherein in the first position the bone anchor retaining member has a generally convex shape.

3. The apparatus of claim 2, wherein in the second position the bone anchor retaining member has a generally concave shape.

4. The apparatus of claim 2, wherein in the second position the bone anchor retaining member has a generally convex shape.

5. The apparatus of claim 1, wherein the cavity is substantially coaxial with the aperture.

6. The apparatus of claim 1, wherein the bone anchor retaining member comprises a plurality of surfaces extending towards a center of the aperture, wherein a distance between the plurality of surfaces in the second position is less than a distance between the plurality of surfaces in the first position.

7. The apparatus of claim 6, wherein the transition comprises a third position between the first position and the second position, wherein in the third position a distance between the plurality of surfaces is less than both the distance between the plurality of surfaces in the first position and the distance between the plurality of surfaces in the second position.

8. The apparatus of claim 1, wherein the bone fixation plate comprises a generally part-spherical surface adjacent to the at least one aperture and the top surface.

9. The apparatus of claim 8, wherein the bone fixation plate comprises a generally part-cylindrical surface adjacent to the at least one aperture and the bottom surface.

10. The apparatus of claim 1, further comprising the bone anchor, wherein a width of the aperture is substantially equal to a width of a portion of the bone anchor that is adjacent to the aperture when the bone anchor is advanced fully into the plate.

11. The apparatus of claim 1, further comprising the bone anchor, wherein a width of the aperture is greater than a width of a portion of the bone anchor that is adjacent to the aperture when the bone anchor is advanced fully into the plate.

12. The apparatus of claim 1, wherein a width of the cavity is substantially equal to a width of the bone anchor retaining member.

13. The apparatus of claim 1, wherein a width of the cavity is greater than a width of the bone anchor retaining member.

14. The apparatus of claim 1, further comprising the bone anchor, wherein the bone anchor is a bone screw comprising:
a head configured for articulation with the bone fixation plate;
a shoulder in communication with the head, the shoulder configured to contact the bone anchor retaining member when the member is in the first position;
a groove in communication with the shoulder, the groove configured to receive the bone anchor retaining member when the member is in the second position; and
a threaded shank in communication with the groove,
wherein a width of the shoulder is greater than a width of the threaded shank.

15. The apparatus of claim 1, wherein the bone anchor retaining member comprises a single piece of solid construction.

16. The apparatus of claim 1, wherein the bone anchor retaining member is formed from an elastic material.

17. A bone fixation apparatus, comprising:
a bone fixation plate comprising a top surface, a bottom surface, and at least one aperture between the top surface and the bottom surface for permitting partial passage of a bone anchor through the plate, the bone fixation plate further comprising a cavity formed between the top surface and the bottom surface and substantially coaxial with the aperture; and
a ring-shaped member within the cavity, the ring-shaped member configured to permit partial passage of the bone anchor and subsequently resist back out of the bone anchor from the member and the bone fixation plate.

18. The bone fixation apparatus of claim 17, wherein the ring-shaped member is configured to transition from a first, convex position to a second, concave position in response to interaction of the member with the bone anchor, wherein in the first position the bone anchor retaining member permits partial passage of the at least one bone anchor through the member and in the second position the member resists back out of the bone anchor.

19. The bone fixation apparatus of claim 17, wherein the ring-shaped member has a flat cross-section configured to deform in order to permit partial passage of the bone anchor and reform subsequent to said partial passage in order to resist back out of the bone anchor.

20. The bone fixation apparatus of claim 17, wherein the bone fixation plate comprises an attachable cover member forming the bottom surface of the bone fixation plate, wherein the cover member is substantially co-axial with the cavity and the ring-shaped member and is configured to prevent the ring-shaped member from ejecting from the cavity.

21. A bone fixation apparatus, comprising:
a bone fixation plate comprising a top surface, a bottom surface, and at least one aperture between the top surface and the bottom surface for permitting partial passage of a bone anchor through the plate, the bone fixation plate further comprising a cavity formed between the top surface and the bottom surface and substantially coaxial with the aperture; and
a bone anchor retaining member within the cavity, the member forming a plurality of peaks and valleys in top and bottom surfaces of the member and configured to transition from a first, convex position to a second, convex position in response to interaction of the member with the bone anchor, wherein in the first position the bone anchor retaining member permits partial passage of the at least one bone anchor through the member and in the second position the member resists back out of the bone anchor.

22. A method for bone fixation, comprising:
advancing a bone anchor through a bone fixation assembly and into bone;
encountering resistance to the advancing before the bone anchor is advanced fully into the bone, wherein the resistance is attributable to the bone fixation assembly;
further advancing the bone anchor into the bone to overcome the resistance; and
subsequent to the further advancing, resisting back out of the bone anchor from the bone fixation assembly.

23. The method of claim 22, wherein encountering resistance to the advancing comprises contacting a portion of the bone fixation assembly with a shoulder of the bone anchor.

24. A bone fixation apparatus, comprising:
means housed within a bone fixation plate for resisting advancement of a bone anchor before the bone anchor is advanced fully into the bone, wherein upon further advancement of the bone anchor through the bone fixation plate, the means housed within the bone fixation plate further comprises means for resisting back out of the bone anchor from the bone fixation plate.

25. The bone fixation apparatus of claim 24, further comprising means attachable to the bone fixation plate for preventing the means housed within the bone fixation plate from ejecting from the housing.
